(19) United States
(12) Patent Application Publication
(10) Pub. No.: US 2015/0153380 A1
(43) Pub. Date: Jun. 4, 2015

(54) METHOD AND SYSTEM FOR ESTIMATING MULTIPLE MODES OF MOTION

(71) Applicant: INVENSENSE, INC., San Jose, CA (US)

(72) Inventors: Mostafa Elhoushi, Calgary (CA); Jacques Georgy, Calgary (CA); Aboelmagd Noureldin, Calgary (CA)

(21) Appl. No.: 14/528,868

(22) Filed: Oct. 30, 2014

Related U.S. Application Data
(60) Provisional application No. 61/897,711, filed on Oct. 30, 2013.

(51) Int. Cl. G01P 15/14

(52) U.S. Cl. CPC G01P 15/14

(57) ABSTRACT

A method and system for determining the mode of motion or conveyance of a device, the device being within a platform (e.g., a person, vehicle, or vessel of any type). The device can be strapped or non-strapped to the platform, and where non-strapped, the mobility of the device may be constrained or unconstrained within the platform and the device may be moved or tilted to any orientation within the platform, without degradation in performance of determining the mode of motion. This method can utilize measurements (readings) from sensors in the device (such as for example, accelerometers, gyroscopes, etc.) whether in the presence or in the absence of navigational information updates (such as, for example, Global Navigation Satellite System (GNSS) or WiFi positioning). The present method and system may be used in any one or both of two different phases, a model building phase or a model utilization phase.

### CROSS-REFERENCE TO RELATED APPLICATION

This application claims priority from U.S. Ser. No. 61/897,711, entitled “METHOD AND APPARATUS FOR ESTIMATING MULTIPLE MODES OF MOTION,” and filed on Oct. 30, 2013, the entire contents of which are incorporated herein as if set forth in full.

### TECHNICAL FIELD

The present disclosure relates to a method and system for estimating multiple modes of motion or conveyance for a device, wherein the device is within a platform (such as for example a person, vehicle, or vessel of any type), wherein the device can be strapped or non-strapped to the platform, and wherein in case of non-strapped the mobility of the device may be constrained or unconstrained within the platform.

### BACKGROUND

Inertial navigation of a platform is based upon the integration of specific forces and angular rates measured by inertial sensors (e.g. accelerometer, gyroscopes) by a device containing the sensors. In general, the device is positioned within the platform and commonly strapped to the platform. Such measurements from the device may be used to determine the position, velocity and attitude of the device and/or the platform.

The platform may be a motion-capable platform that may be temporarily stationary. Some of the examples of the platforms may be a person, a vehicle or a vessel of any type. The vessel may be land-based, marine or airborne.

Alignment of the inertial sensors within the platform (and with the platform's forward, transversal and vertical axis) is critical for inertial navigation. If the inertial sensors, such as accelerometers and gyroscopes are not exactly aligned with the platform, the positions and attitude calculated using the readings of the inertial sensors will not be representative of the platform. Fixing the inertial sensors within the platform is thus a requirement for navigation systems that provide high accuracy navigation solutions.

For strapped systems, one means for ensuring optimal navigation solutions is to utilize careful manual mounting of the inertial sensors within the platform. However, portable navigation devices (or navigation-capable devices) are able to move whether constrained or unconstrained within the platform (such as for example a person, vehicle or vessel), so careful mounting is not an option.

For navigation, mobile/smart phones are becoming very popular as they come equipped with Assisted Global Positioning System (AGPS) chipsets with high sensitivity capabilities to provide absolute positions of the platform even in some environments that cannot guarantee clear line of sight to satellite signals. Deep indoor or challenging outdoor navigation or localization incorporates cell tower identification (ID) or, if possible, cell towers trilateration for a position fix where AGPS solution is unavailable. Despite these two positioning methods that already come in many mobile devices, accurate indoor localization still presents a challenge and fails to satisfy the accuracy demands of today's location based services (LBS). Additionally, these methods may only provide the absolute heading of the platform without any information about the device's heading.

Many mobile devices, such as mobile phones, are equipped with Micro Electro Mechanical System (MEMS) sensors that are used predominantly for screen control and entertainment applications. These sensors have not been broadly used to date for navigation purposes due to very high noise, large random drift rates, and frequently changing orientations with respect to the carrying platform.

Magnetometers are also found within many mobile devices. In some cases, it has been shown that a navigation solution using accelerometers and magnetometers may be possible if the user is careful enough to keep the device in a specific orientation with respect to their body, such as when held carefully in front of the user after calibrating the magnetometer.

There is a need for a navigation solution capable of accurately utilizing measurements from a device within a platform to determine the navigation state of the device/platform without any constraints on the platform (i.e. in indoor or outdoor environments), the mode of motion/conveyance, or the mobility of the device. The estimation of the position and attitude of the platform has to be independent of the mode of motion/conveyance (such as for example walking, running, cycling, in a vehicle, bus, or train among others) and usage of the device (e.g. the way the device is put or moving within the platform during navigation). In the above scenarios, it is required that the device provide seamless navigation. This again highlights the key importance of obtaining the mode of motion/conveyance of the device as it is a key factor to enable portable navigation devices without any constraints.

Thus methods of determining the mode of motion/conveyance are required for navigation using devices, wherein mobility of the device may be constrained or unconstrained within the platform.

In addition to the above mentioned application of portable devices (that involves a full navigation solution including position, velocity and attitude, or position and attitude), there are other applications (that may involve estimating a fill navigation solution, or an attitude only solution or an attitude and velocity solution) where the method to estimate the mode of motion/conveyance is needed for enhancing the user experience and usability, and may be applicable in a number of scenarios.

### SUMMARY

The present disclosure relates to a method and system for determining the mode of motion or conveyance of a device, wherein the device is within a platform (such as for example a person, vehicle, or vessel of any type), wherein the device can be strapped or non-strapped to the platform, wherein in case of non-strapped the mobility of the device may be constrained or unconstrained within the platform. In case of non-strapped, the device may be moved or tilted to any orientation within the platform and still provide the mode of motion or conveyance without degrading the performance of determining the mode. The present method can utilize measurements (readings) from sensors in the device (such as for example, accelerometers, gyroscopes, barometer, etc.) whether in the presence or in the absence of navigational information updates (such as, for example, Global Navigation Satellite System (GNSS) or WiFi positioning).

The present method and system may be used in any one or both of two different phases. In sonic embodiments, the first phase only is used. In some other embodiments, the second phase only is used. In a third group of embodiments, the first phase is used, and then the second phase is used. It is understood that the first and second phases need not be used in sequence. The first phase, referred to as the “model-building phase”, is a model building or training phase done offline to obtain a classifier model for the determination of the mode of motion/conveyance as a function of different parameters and features that represent motion dynamics or stationarity. Features extraction and classification techniques may be used for this phase. In the second phase, referred to as “model utilization phase”, feature extraction and an obtained classifier model are used to determine the mode of motion/conveyance. The features may be obtained from sensors readings from the sensors in the system. This second phase may be the more frequent usage of the present method and system for a variety of applications.

In one embodiment, in order to be able to achieve a classifier model that can determine the mode of motion or conveyance regardless of: (i) the device usage or use case (in hand, pocket, on ear, in belt, . . . ), (ii) the device orientation, (iii) the platform or user's features, varying motion dynamics, speed, . . . , the model may be built with a large data set of collected trajectories covering all the above varieties in addition to covering all the modes of motion or conveyance to be determined. The present method and system may use different features that represent motion dynamics or stationarity to be able to discriminate the different modes of motion or conveyance to be determined.

During the model-building phase, a group of people collect the datasets used for building the model (datasets consist of sensors readings) with all the modes of motion or conveyance to be determined (including those on foot, in vehicle or vessel) and covering all the varieties mentioned in the previous paragraph. During model-building, for each epoch of collected sensor readings, a reference mode of motion or conveyance is assigned and the used features that represent motion dynamics or stationarity are calculated, stored and then fed to the model-building technique.

In the more frequent usage of the present method and system, i.e. the “model utilization phase”, the classifier model can be used to determine the mode of motion or conveyance from the different features that represent motion dynamics or stationarity used as input to the model, where these features are obtained from sensors readings.

In some embodiments, the application using the present method and system can use: (i) the model-building phase only, (ii) the model utilization phase, or (iii) both model-building phase and then the model utilization phase.

In some embodiments, the output of the classifier model is a determination of the mode of motion or conveyance. In some other embodiments, the output of the classifier is a determination of the probability of each mode of motion or conveyance.

In some embodiments, a routine for feature selection to choose the most suitable subset of features may be used on the feature vector, before training or evaluating a classifier model, in order to improve classification results.

In some embodiments, a routine for feature transformation may be used on the feature vector, before training or evaluating a classifier model, in order to improve classification results. Feature transformation aims to obtain a mathematical transformation of the feature vector to create a new feature vector, the new feature vector being more representable of the mode of motion or conveyance.

In some embodiments, a routine can run after the model usage in determining mode of motion or conveyance to refine the results based on other information, if such information is available. For example, if GNSS information is available with good accuracy measure, then GNSS information such as velocity can be used to either choose or discard some modes of motion or conveyance.

In some embodiments, a routine can run after the model usage in determining mode of motion or conveyance to refine the results based on previous history of determined mode of motion or conveyance. Any type of filtering, averaging or smoothing may be used. An example is to use a majority over a window of history data. Furthermore, techniques such as hidden Markov Models may be used.

In some embodiments, a routine for meta-classification methods could be used where several classifiers (of the same type or of different types) are trained over the same training data set, or trained on different subsets of the training data set, or trained using other classifier outputs as additional features, then evaluated, and their results combined in various ways.

For the model-building phase, in order to run the technique used to build the model, any machine or an apparatus which is capable of processing can be used, where the model-building technique can be run and outputs the model for determining the mode of motion or conveyance.

For the present method and system to perform its functionality at least accelerometer(s) and gyroscope(s) are used. In one embodiment, the system includes at least a tri-axial accelerometer and at least a tri-axial gyroscope, which may be used as the sole sensors. In some embodiments, in addition to the above-mentioned inertial sensors the system may include additional types of sensors such as for example magnetometers, barometers or any other type of additional sensors, any of the available sensors may be used. The system may also include a source of obtaining absolute navigational information (such as GNSS, WiFi, RFID, Zigbee, Cellular based localization among others), any other positioning system, or combination of systems may be included as well.

In some embodiments, the system may also include processing means. In some of these embodiments, the sensors in the system are in the same device or module as the processing means. In some other embodiments, the sensors included in the system may be contained in a separate device or module other than the device or module containing the processing means, the two devices or modules may communicate through a wired or a wireless mean of communication.

In some embodiments, in the model-building, the system (whether in one or more device(s) or module(s)) may be used for any one of the following: (i) data collection and logging (e.g., saving or storing) while the model-building technique runs on another computing machine, (ii) data reading and processing the model-building technique, (iii) data collection, logging (this means saving or storing), and processing the model-building technique.

In some embodiments, in the model usage to determine the mode of motion or conveyance, the aforementioned system (whether in one or more device(s) or module(s)) may be used for any one of the following: (i) data collection and logging (this means saving or storing) while using the model for determining the mode of motion or conveyance runs on another computing machine, (ii) data reading and using the model for determining the mode of motion or conveyance, (iii) data collection, logging (this means saving or storing), and using the model for determining the mode of motion or conveyance.

Broadly stated, in some embodiments, a method is provided for determining the mode of motion of a device, the device being within a platform and strapped or non-strapped to the platform, where non-strapped, mobility of the device may be constrained or unconstrained within the platform, the device having sensors capable of providing sensor readings, the method comprising the steps of: a) obtaining features that represent motion dynamics or stationarity from the sensor readings; and b) using the features to: (i) build a model capable of determining the mode of motion, (ii) utilize a model built to determine the mode of motion, or (iii) build a model capable of determining the mode of motion of the device, and utilizing said model built to determine the mode of motion.

Broadly stated, in some embodiments, a system is provided for determining the mode of motion of a device, the device being within a platform, the system comprising: a) the device strapped or non-strapped to the platform, where non-strapped, the mobility of the device may be constrained or unconstrained within the platform, the device comprising: i) sensors capable of providing sensor readings; and b) a processor programmed to receive the sensor readings, and operative to: i) obtain features that represent motion dynamics or stationarity from the sensor readings; and ii) use the features to: (A) build a model capable of determining the mode of motion, (B) utilize a model built to determine the mode of motion, or (C) build a model capable of determining the mode of motion and utilizing said model built to determine the mode of motion.

Broadly stated, in some embodiments, a method is provided for determining the mode of motion of a device, the device being within a platform and strapped or non-strapped to the platform, where non-strapped, mobility of the device may be constrained or unconstrained within the platform, the device having sensors capable of providing sensor readings, the method comprising the steps of: a) obtaining the sensor readings for a plurality of modes of motion; b) obtaining features that represent motion dynamics or stationarity from the sensor readings; c) indicating reference modes of motion corresponding to the sensor readings and the features; d) feeding the features and the reference modes of motion to a technique for building a model capable of determining the mode of motion; and e) running the technique.

Broadly stated, in some embodiments, a system is provided for determining the mode of motion of a device, the device being within a platform, the system comprising: a) the device strapped or non-strapped to the platform, and where non-strapped, the mobility of the device may be constrained or unconstrained within the platform, the device comprising: i) sensors capable of providing sensor readings; and b) a processor operative to: i) obtain the sensor readings for a plurality of modes of motion; ii) obtain features that represent motion dynamics or stationarity from the sensor readings; iii) indicate reference modes of motion corresponding to the sensor readings and the features; iv) feed the features and the reference modes of motion to a technique for building a model capable of determining the mode of motion; and v) run the technique.

Broadly stated, in some embodiments, a method is provided for determining the mode of motion of a device, the device being within a platform and strapped or non-strapped to the platform, where non-strapped, mobility of the device may be constrained or unconstrained within the platform, the device having sensors capable of providing sensor readings, the method comprising the steps of: a) obtaining the sensor readings; b) obtaining features that represent motion dynamics or stationarity from the sensor readings; c) passing the features to a model capable of determining the mode of motion from the features; and d) determining an output mode of motion from the model.

Broadly stated, in some embodiments, a system is provided for determining the mode of motion of a device, the device being within a platform, the system comprising: a) the device strapped or non-strapped to the platform, where non-strapped, the mobility of the device may be constrained or unconstrained within the platform, the device comprising: i) sensors capable of providing sensor readings; and b) a processor operative to: i) obtain the sensor readings; ii) obtain features that represent motion dynamics or stationarity from the sensor readings; iii) pass the features to a model capable of determining the mode of motion from the features; and iv) determine an output mode of motion from the model.

### DESCRIPTION OF THE DRAWINGS

FIG. 1 is a flow chart showing: (a) an embodiment of the method using the model building phase, (b) an embodiment of the method using the model utilization phase, and (c) an embodiment of the method using both the model building phase and the model utilization phase.

FIG. 2 is a flow chart showing an example of the steps for the model building phase.

FIG. 3 is a flow chart showing an example of the steps for the model utilization phase.

FIG. 4 is a block diagram depicting a first example of the device according to embodiments herein.

FIG. 5 is a block diagram depicting a second example of the device according to embodiments herein.

FIG. 6 shows an overview of one embodiment for determining the mode of motion.

FIG. 7 shows an exemplary axes frame of portable device prototype.

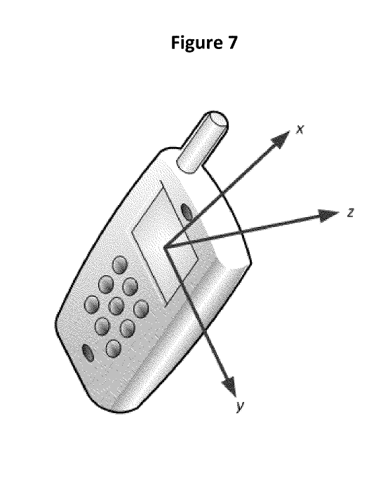

### DETAILED DESCRIPTION

The present disclosure relates to a method and system for determining the mode of motion or conveyance of a device, wherein the device is within a platform (such as for example a person, vehicle, or vessel of any type), wherein the device can be strapped or non-strapped to the platform, wherein in case of non-strapped the mobility of the device may be constrained or unconstrained within the platform. In case of non-strapped, the device may be moved or tilted to any orientation within the platform while providing the mode of motion or conveyance without degrading the performance of determining the mode. This method can utilize measurements (readings) from sensors in the device (such as for example, accelerometers, gyroscopes, barometer, etc.) whether in the presence or in the absence of absolute navigational information (such as, for example, Global Navigation Satellite System (GNSS) or WiFi positioning).

The device is “strapped”, “strapped down”, or “tethered” to the platform when it is physically connected to the platform in a fixed manner that does not change with time during navigation, in the case of strapped devices, the relative position and orientation between the device and platform does not change with time during navigation. The device is “non-strapped”, or “non-tethered” when the device has some mobility relative to the platform (or within the platform), meaning that the relative position or relative orientation between the device and platform may change with time during navigation. The device may be “non-strapped” in two scenarios: where the mobility of the device within the platform is “unconstrained”, or where the mobility of the device within the platform is “constrained”. One example of “unconstrained” mobility may be a person moving on foot and having a portable device such as a smartphone in the their hand for texting or viewing purposes (hand may also move), at their ear, in hand and dangling/swinging, in a belt clip, in a pocket, among others, where such use cases can change with time and even each use case can have a changing orientation with respect to the user. Another example where the mobility of the device within the platform is “unconstrained” is a person in a vessel or vehicle, where the person has a portable device such as a smartphone in the their hand for texting or viewing purposes (hand may also move), at their ear, in a belt clip, in a pocket, among others, where such use cases can change with time and even each use case can have a changing orientation with respect to the user. An example of “constrained” mobility may be when the user enters a vehicle and puts the portable device (such as smartphone) in a rotation-capable holder or cradle. In this example, the user may rotate the holder or cradle at any time during navigation and thus may change the orientation of the device with respect to the platform or vehicle.

Absolute navigational information is information related to navigation and/or positioning and are provided by “reference-based” systems that depend upon external sources of information, such as for example Global Navigation Satellite Systems (GNSS). On the other hand, self-contained navigational information is information related to navigation and/or positioning and is provided by self-contained and/or “non-reference based” systems within a device/platform, and thus need not depend upon external sources of information that can become interrupted or blocked. Examples of self-contained information are readings from motion sensors such as accelerometers and gyroscopes.

The present method and system may be used in any one or both of two different phases. In some embodiments, only the first phase is used. In some other embodiments, only the second phase is used. In a third group of embodiments, the first phase is used, and then the second phase is used. It is understood that the first and second phases need not be used in sequence. The first phase, referred to as the “model-building phase”, is a model building or training phase done offline to obtain a classifier model for the determination of the mode of motion or conveyance as a function of different parameters and features that represent motion dynamics or stationarity. Features extraction and classification techniques may be used for this phase. In the second phase, referred to as the “model utilization phase”, feature extraction and an obtained classifier model are used to determine the mode of motion/conveyance. The features may be obtained from sensor readings from the sensors in the system. This second phase may be the more frequent usage of the present method and system for a variety of applications.

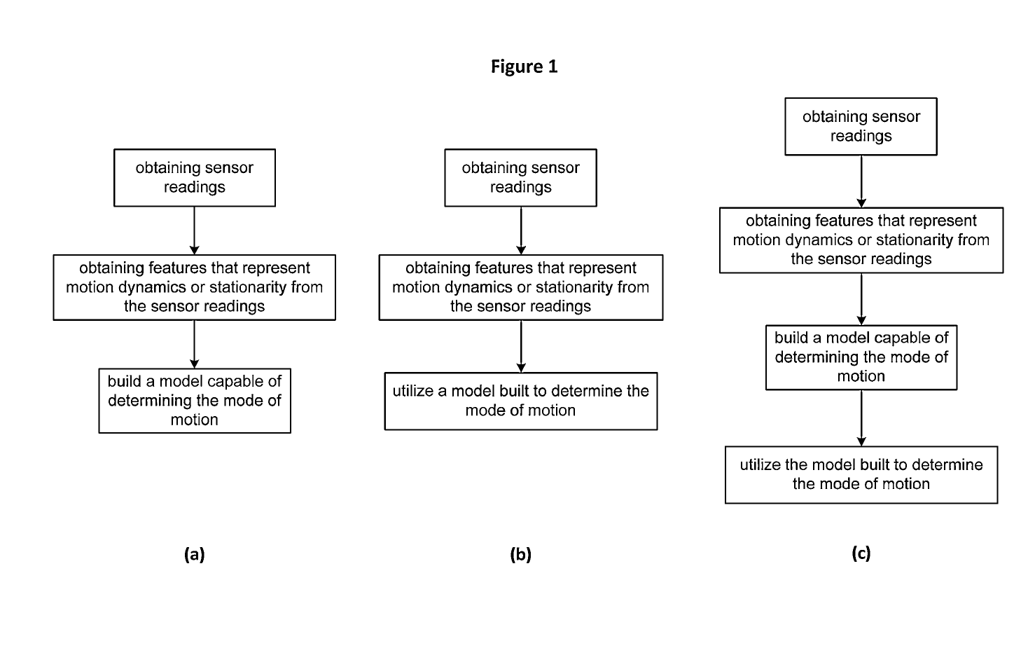

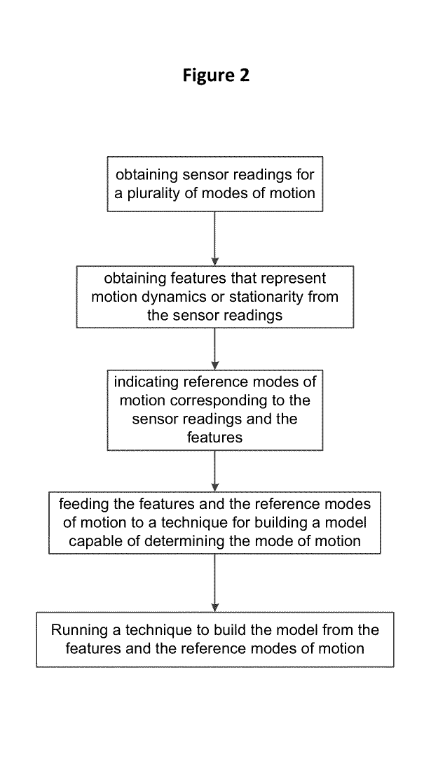

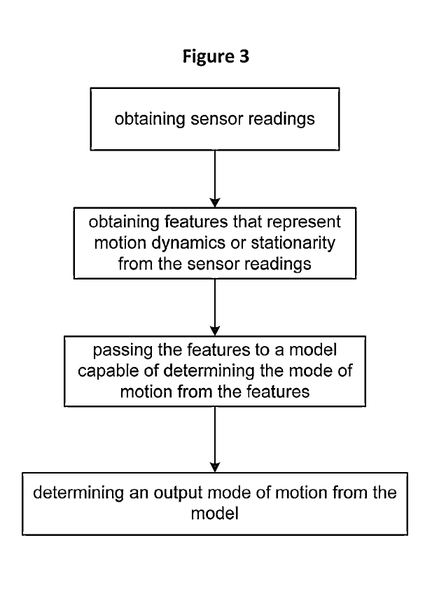

The first phase, which is the model building phase, is depicted in FIG. 1(a); the second phase, which is the model utilization phase, is depicted in FIG. 1(b); and an embodiment using both model-building and model utilization phases is depicted in FIG. 1(c). Having regard to FIG. 2, the steps of an embodiment of the model building phase are shown. Having regard to FIG. 3, the steps of an embodiment of the model utilization phase are shown.

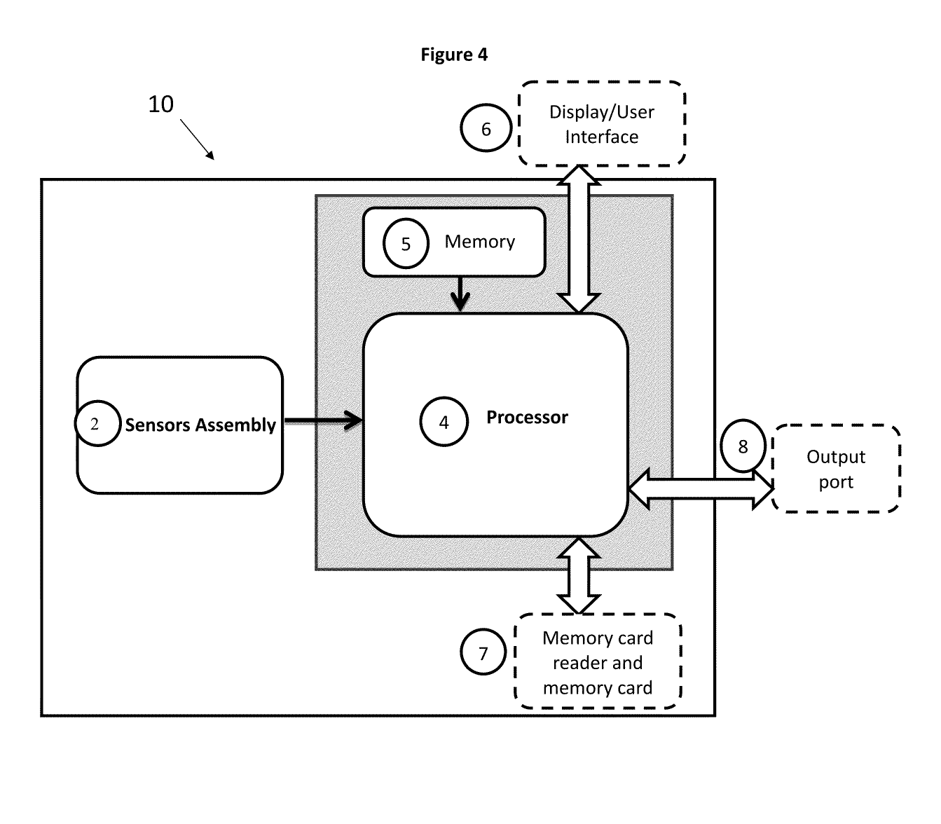

Having regard to FIG. 4, the present device 10 may include a self-contained sensor assembly 2, capable of obtaining or generating “relative” or “non-reference based” readings relating to navigational information about the moving device, and producing an output indicative thereof. In one embodiment, the sensor assembly 2 may, for example, include at least accelerometers for measuring accelerations, and gyroscopes for measuring rotation rates. In another embodiment, the sensor assembly 2 may, for example, include at least a tri-axial accelerometer for measuring accelerations, and a tri-axial gyroscope for measuring rotation rates. In yet another embodiment, the sensor assembly 2 may, optionally, include other self-contained sensors such as, without limitation a three dimensional (3D) magnetometer, for measuring magnetic field strength for establishing heading; a barometer, for measuring pressure to establish altitude; or any other sources of either self-contained and/or “relative” navigational information.

In some embodiments, the present device 10 may comprise at least one processor 4 coupled to receive the sensor readings from the sensor assembly 2. In some embodiments, the present device 10 may comprise at least one memory 5. Optionally, the device 10 may include a display or user interface 6. It is contemplated that the display 6 may be part of the device 10, or separate therefrom (e.g., connected wired or wirelessly thereto). Optionally, the device 10 may include a memory device/card 7. It is contemplated that the memory device/card 7 may be part of the device 10, or separate therefrom (e.g., connected wired or wirelessly thereto). Optionally, the device 10 may include an output port 8.

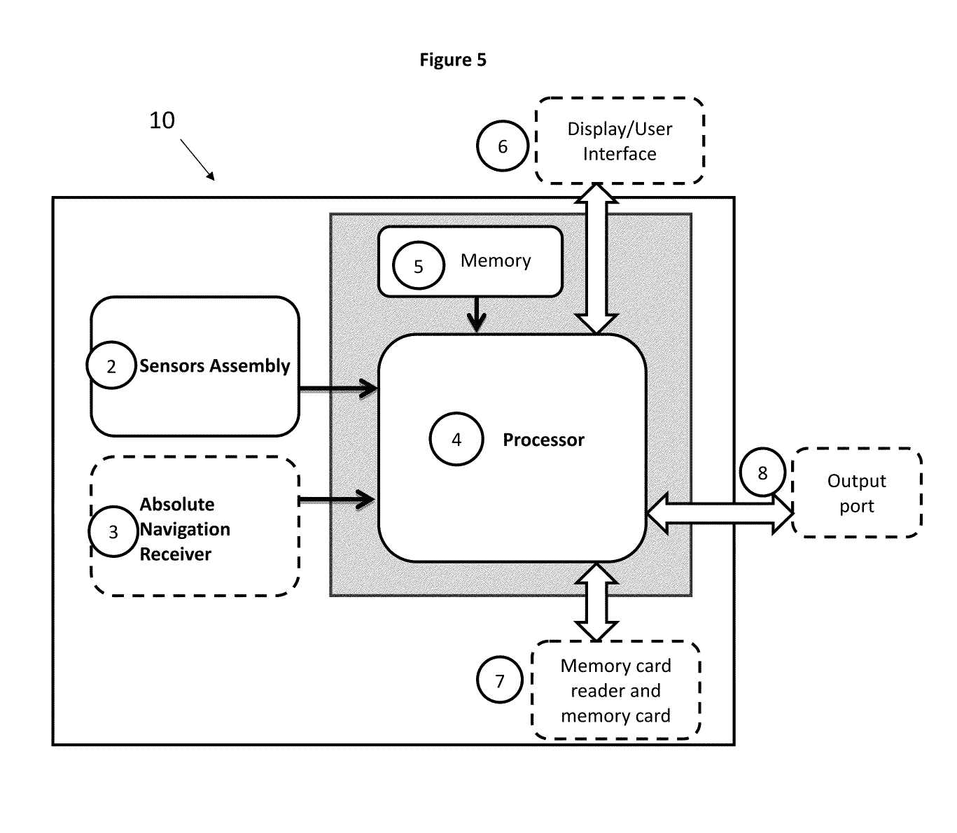

Having regard to FIG. 5, the present device 10 may include a self-contained sensor assembly 2, capable of obtaining or generating “relative” or “non-reference based” readings relating to navigational information about the moving device, and producing an output indicative thereof. In one embodiment, the sensor assembly 2 may, for example, include at least one accelerometer, for measuring acceleration rates. In another embodiment, the sensor assembly 2 may, for example, include at least tri-axial accelerometer, for measuring acceleration rates. In yet another embodiment, the sensor assembly 2 may, optionally, include other self-contained sensors such as, without limitation, a gyroscope, for measuring turning rates of the of the device; a three dimensional (3D) magnetometer, for measuring magnetic field strength for establishing heading; a barometer, for measuring pressure to establish altitude; or any other sources of “relative” navigational information.

The present training device 10 may also include a receiver 3 capable of receiving “absolute” or “reference-based” navigation information about the device from external sources, such as satellites, whereby receiver 3 is capable of producing an output indicative of the navigation information. For example, receiver 3 may be a GNSS receiver capable of receiving navigational information from GNSS satellites and converting the information into position and velocity information about the moving device. The GNSS receiver may also provide navigation information in the form of raw measurements such as pseudoranges and Doppler shifts. The GNSS receiver might operate in one of different modes, such as, for example, single point, differential, RTK, PPP, or using wide area differential (WAD) corrections (e.g. WAAS).

In some embodiments, the present device 10 may include at least one processor 4 coupled to receive the sensor readings from the sensor assembly 2, and the absolute navigational information output from the receiver 3. In some embodiments, the present device 10 may include at least one memory 5. Optionally, the device 10 may include a display or user interface 6. It is contemplated that the display 6 may be part of the device 10, or separate therefrom (e.g., connected wired or wirelessly thereto). Optionally, the device 10 may include a memory device/card 7. It is contemplated that the memory device/card 7 may be part of the device 10, or separate therefrom (e.g., connected wired or wirelessly thereto). Optionally, the device 10 may include an output port 8.

In one embodiment, in order to be able to achieve a classifier model that can determine the mode of motion or conveyance regardless of: (i) the device usage or use case (in hand, pocket, on ear, in belt, . . . ), (ii) the device orientation, (iii) the platform or user's features, varying motion dynamics, speed, . . . , the model should be built with a large data set of collected trajectories covering all the above varieties in addition to covering all the modes of motion or conveyance to be determined. The present method and system may use different features that represent motion dynamics or stationarity to be able to discriminate the different modes of motion or conveyance to be determined.

During the model-building phase, the first stage is data collection. A group of people collect the datasets used for building the model (datasets consist of sensors readings) with all the modes of motion or conveyance to be determined (including those on foot, in vehicle or vessel) and covering all the varieties mentioned in the previous paragraph.

During model-building, for each epoch of collected sensor readings, a reference mode of motion or conveyance is assigned and the used features that represent motion dynamics or stationarity are calculated, stored and then fed to the model-building technique. The used features are calculated for each epoch of collected sensor readings in order to be used for building the classifier model. The sensors readings can be used “as is”, or optional averaging, smoothing, or filtering (such as for example low pass filtering) may be performed.

During model-building, the second stage is to feed the collected data to the model building technique, then run it to build and obtain the model. The mode of motion or conveyance is the target output used to build the model, and the features that represent motion dynamics or stationarity constitute the inputs to the model corresponding to the target output. In some embodiments, the model building technique is a classification technique such as for example, decision trees or random forest.

In the more frequent usage of the present method and system, the classifier model is used to determine the mode of motion or conveyance from the different features that represent motion dynamics or stationarity used as input to the model, where these features are obtained from sensors readings.

In some embodiments, the application using the present method and system can use: (i) the model-building phase only, (ii) the model utilization phase only, or (iii) both model-building phase then model utilization phase.

In some embodiments, the output of the classifier model is a determination of the mode of motion or conveyance. In some other embodiments, the output of the classifier is a determination of the probability of each mode of motion or conveyance.

In some embodiments, an optional routine for feature selection to choose the most suitable subset of features may be used on the feature vector, before training or evaluating a classifier, in order to obtain better classification results.

In some embodiments, an optional routine for feature transformation may be used on the feature vector, before training or evaluating a classifier, in order to obtain better classification results. Feature transformation aims to obtain a mathematical transformation of the feature vector to create a new feature vector which is better and more representable of the mode of motion or conveyance.

In some embodiments, an optional routine can run after the model usage in determining mode of motion or conveyance to refine the results based on other information, if such information is available. For example, if GNSS information is available with good accuracy measure, then GNSS information such as velocity can be used to either choose or discard some modes of motion or conveyance.

In some embodiments, an optional routine can run after the model usage in determining mode of motion or conveyance to refine the results based on the previous history of determined mode of motion or conveyance. Any type of filtering, averaging or smoothing may be used. An example is to use a majority over a window of history data. Furthermore, techniques such as hidden Markov Models may be used.

In some embodiments, an optional routine for meta-classification methods could be used where several classifiers (of the same type or of different types) are trained over the same training data set, or trained on different subsets of the training data set, or trained using other classifier outputs as additional features, then evaluated, and their results combined in various ways. Some examples of meta-classification methods which may be used are: boosting, bagging, plurality voting, cascading, stacking with ordinary-decision trees, stacking with meta-decision trees, or stacking using multi-response linear regression.

For the model-building, in order to run the technique used to build the model, any machine or an apparatus which is capable of processing can be used, where the model-building technique can be run and outputs a model for determining the mode of motion or conveyance.

For the present method and system to perform its functionality, sensors comprising at least accelerometer(s) and gyroscope(s) are needed. In one embodiment, the system may include inertial sensors having at least a tri-axial accelerometer and at least a tri-axial gyroscope, which may be used as the sole sensors. In some embodiments, in addition to the above-mentioned inertial sensors, the system may include additional types of sensors such as for example magnetometers, barometers or any other type of additional sensors. Any of the available sensors may be used. The system may also include a source of obtaining absolute navigational information (such as GNSS, WiFi, RFID, Zigbee, Cellular based localization among others), any other positioning system, or combination of systems may be included as well.

In some embodiments, the system may also include processing means. In some of these embodiments, the sensors in the system are in the same device or module as the processing means. In some other embodiments, the sensors included in the system may be contained in a separate device or module other than the device or module containing the processing means, the two devices or modules may communicate through a wired or a wireless mean of communication. In the embodiments that include a source of absolute navigational information, said source may be in the same device or module including the sensors or it may be in another device or module that is connected wirelessly or wired to the device including the sensors.

In some embodiments, in the model-building phase, the system (whether in one or more device(s) or module(s)) may be used for any one of the following: (i) data collection and logging (this means saving or storing) while the model-building technique runs on another computing machine, (ii) data reading and processing the model-building technique, (iii) data collection, logging (this means saving or storing), and processing the model-building technique.

In some embodiments, in the model utilization phase to determine the mode of motion or conveyance, the system (whether in one or more device(s) or module(s)) may be used for any one of the following: (i) data collection and logging (e.g., means saving or storing) while using the model for determining the mode of motion or conveyance runs on another computing machine, (ii) data reading and using the model for determining the mode of motion or conveyance, (iii) data collection, logging (this means saving or storing), and using the model for determining the mode of motion or conveyance.

Optionally, the present method and system may be used with any navigation system such as for example: inertial navigation system (INS), absolute navigational information systems (such as GNSS, WiFi, RFID, Zigbee, Cellular based localization among others), any other positioning system, combination of systems, or any integrated navigation system integrating any type of sensors or systems and using any type of integration technique.

When the method and system presented herein is combined in any way with a navigation solution, this navigation solution can use any type of state estimation or filtering techniques. The state estimation technique can be linear, nonlinear or a combination thereof. Different examples of techniques used in the navigation solution may rely on a Kalman filter, an Extended Kalman filter, a non-linear filter such as a particle filter, or an artificial intelligence technique such as Neural Network or Fuzzy systems. The state estimation technique used in the navigation solution can use any type of system and/or measurement models. The navigation solution may follow any scheme for integrating the different sensors and systems, such as for example loosely coupled integration scheme or tightly coupled integration scheme among others. The navigation solution may utilize modeling (whether with linear or nonlinear, short memory length or long memory length) and/or automatic calibration for the errors of inertial sensors and/or the other sensors used.

### Contemplated Embodiments

It is contemplated that the method and system presented above can be used with a navigation solution that may optionally utilize automatic zero velocity updates and inertial sensors bias recalculations, non-holonomic updates module, advanced modeling and/or calibration of inertial sensors errors, derivation of possible measurements updates for them from GNSS when appropriate, automatic assessment of GNSS solution quality and detecting degraded performance, automatic switching between loosely and tightly coupled integration schemes, assessment of each visible GNSS satellite when in tightly coupled mode, and finally possibly can be used with a backward smoothing module with any type of backward smoothing technique and either running in post mission or in the background on buffered data within the same mission.

It is further contemplated that the method and system presented above can be used with a navigation solution that is further programmed to run, in the background, a routine to simulate artificial outages in the absolute navigational information and estimate the parameters of another instance of the state estimation technique used for the solution in the present navigation module to optimize the accuracy and the consistency of the solution. The accuracy and consistency is assessed by comparing the temporary background solution during the simulated outages to a reference solution. The reference solution may be one of the following examples: the absolute navigational information (e.g. GNSS), the forward integrated navigation solution in the device integrating the available sensors with the absolute navigational information (e.g. GNSS) and possibly with the optional speed or velocity readings, a backward smoothed integrated navigation solution integrating the available sensors with the absolute navigational information (e.g. GNSS) and possibly with the optional speed or velocity readings. The background processing can run either on the same processor as the forward solution processing or on another processor that can communicate with the first processor and can read the saved data from a shared location. The outcome of the background processing solution can benefit the real-time navigation solution in its future run (i.e. real-time run after the background routine has finished running), for example, by having improved values for the parameters of the forward state estimation technique used for navigation in the present module.

It is further contemplated that the method and system presented above can also be used with a navigation solution that is further integrated with maps (such as street maps, indoor maps or models, or any other environment map or model in cases of applications that have such maps or models available), and a map matching or model matching routine. Map matching or model matching can further enhance the navigation solution during the absolute navigation information (such as GNSS) degradation or interruption. In the case of model matching, a sensor or a group of sensors that acquire information about the environment can be used such as, for example, Laser range finders, cameras and vision systems, or sonar systems. These new systems can be used either as an extra help to enhance the accuracy of the navigation solution during the absolute navigation information problems (degradation or absence), or they can totally replace the absolute navigation information in some applications.

It is further contemplated that the method and system presented above can also be used with a navigation solution that, when working either in a tightly coupled scheme or a hybrid loosely/tightly coupled option, need not be bound to utilize pseudorange measurements (which are calculated from the code not the carrier phase, thus they are called code-based pseudoranges) and the Doppler measurements (used to get the pseudorange rates). The carrier phase measurement of the GNSS receiver can be used as well, for example: (i) as an alternate way to calculate ranges instead of the code-based pseudoranges, or (ii) to enhance the range calculation by incorporating information from both code-based pseudorange and carrier-phase measurements, such enhancement is the carrier-smoothed pseudorange.

It is further contemplated that the method and system presented above can also be used with a navigation solution that relies on an ultra-tight integration scheme between GNSS receiver and the other sensors' readings.

It is further contemplated that the method and system presented above can also be used with a navigation solution that uses various wireless communication systems that can also be used for positioning and navigation either as an additional aid (will be more beneficial when GNSS is unavailable) or as a substitute for the GNSS information (e.g. for applications where GNSS is not applicable). Examples of these wireless communication systems used for positioning are, such as, those provided by cellular phone towers and signals, radio signals, digital television signals, Wifi, or Wimax. For example, for cellular phone based applications, an absolute coordinate from cell phone towers and the ranges between the indoor user and the towers may be utilized for positioning, whereby the range might be estimated by different methods among which calculating the time of arrival or the time difference of arrival of the closest cell phone positioning coordinates. A method known as Enhanced Observed Time Difference (E-OTD) can be used to get the known coordinates and range. The standard deviation for the range measurements may depend upon the type of oscillator used in the cell phone, and cell tower timing equipment and the transmission losses. WiFi positioning can be done in a variety of ways that includes but not limited to time of arrival, time difference of arrival, angles of arrival, received signal strength, and fingerprinting techniques, among others; all of the methods provide different level of accuracies. The wireless communication system used for positioning may use different techniques for modeling the errors in the ranging, angles, or signal strength from wireless signals, and may use different multipath mitigation techniques. All the above mentioned ideas, among others, are also applicable in a similar manner for other wireless positioning techniques based on wireless communications systems.

It is further contemplated that the method and system presented above can also be used with a navigation solution that utilizes aiding information from other moving devices. This aiding information can be used as additional aid (that will be more beneficial when GNSS is unavailable) or as a substitute for the GNSS information (e.g. for applications where GNSS based positioning is not applicable). One example of aiding information from other devices may be capable of relying on wireless communication systems between different devices. The underlying idea is that the devices that have better positioning or navigation solution (for example having GNSS with good availability and accuracy) can help the devices with degraded or unavailable GNSS to get an improved positioning or navigation solution. This help relies on the well-known position of the aiding device(s) and the wireless communication system for positioning the device(s) with degraded or unavailable GNSS. This contemplated variant refers to the one or both circumstance(s) where: (i) the device(s) with degraded or unavailable GNSS utilize the methods described herein and get aiding from other devices and communication system, (ii) the aiding device with GNSS available and thus a good navigation solution utilize the methods described herein. The wireless communication system used for positioning may rely on different communication protocols, and it may rely on different methods, such as for example, time of arrival, time difference of arrival, angles of arrival, and received signal strength, among others. The wireless communication system used for positioning may use different techniques for modeling the errors in the ranging and/or angles from wireless signals, and may use different multi path mitigation techniques.

It is contemplated that the method and system presented above can also be used with various types of inertial sensors, other than MEMS based sensors described herein by way of example.

Without any limitation to the foregoing, the embodiments presented above are further demonstrated by way of the following examples. Reference is also made to the following tables presented in Appendix A to this specification in which:

Table 1 shows various modes of motion detected in one embodiment of the present method and system.

Table 2 shows a confusion matrix of the following modes of motion: stairs, elevator, escalator standing, and escalator walking (as described in Example 3-a herein).

Table 3 shows a confusion matrix of the following modes of motion: stairs and escalator moving (as described in Example 3-b).

Table 4 shows a confusion matrix of the following modes of motion: elevator and escalator standing (as described in Example 3-b).

Table 5 shows a confusion matrix of the following modes of motion: walking, running, bicycle, and land-based vessel (as described in Example 4).

Table 6 shows a confusion matrix of GNSS-ignored trajectories for the following modes of motion: walking, running, bicycle, and land-based vessel (as described in Example 4).

Table 7 shows a confusion matrix of the following modes of motion: stationary and non-stationary (as described in Example 5).

Table 8 shows a confusion matrix of GNSS-ignored trajectories for the following modes of motion: stationary and non-stationary (as described in Example 5).

Table 9 shows a confusion matrix of the following modes of motion: stationary and standing on moving walkway (as described in Example 6).

Table 10 shows a confusion matrix of the following modes of motion: walking and walking on moving walkway (as described in Example 6).

Table 11 shows a confusion matrix of the following modes of motion: walking and walking in land-based vessel (as described in Example 7).

### EXAMPLES

### Example 1

### Demonstration of Determining Multiple Modes of Motion or Conveyance

This example is a demonstration of the present method and system to determine mode of motion or conveyance of a device within a platform, regardless of the type of platform (person, vehicle, vessel of any type), regardless of the dynamics of the platform, regardless of the use case of the device is, regardless of what orientation the device is in, and regardless whether GNSS coverage exists or not. By the term “use case”, it is meant the way the portable device is held or used, such as for example, handheld (texting), held in hand still by side of body, dangling, on ear, in pocket, in belt holder, strapped to chest, arm, leg, or wrist, in backpack or in purse, on seat, or in car holder.

Examples of the motion modes which can be detected by the present method and system are:
Walking
Running/Jogging
Crawling
Fidgeting
Upstairs/Downstairs
Uphill/Downhill/Tilted Hill
Cycling
Land-based Vessel
   Car
      On Platform
      Sitting
   Bus: Within City—Between Cities
      On Platform
      Sitting
      Standing
      Walking
   Train: Between Cities—Light Rail Transit—Streetcar (also known as Tram)—Rapid Rail Transit (also known as Metro or Subway
      On Platform
      Sitting
      Standing
      Walking
Airborne Vessel
   On Platform
   Sitting
   Standing
   walking
Marine Vessel
   On Platform
   Sitting
   Standing
   Walking
Elevator Up/Down
Escalator Up/Down
   Standing
   Walking
Moving Walkway (Conveyor Belt)
   Standing
   Walking
Stationary
   Ground
      On Platform
      Sitting
      Standing
   Land-based Vessel
      Car
         On Platform
         Sitting
      Bus: Within City—Between Cities
         On Platform
         Sitting
         Standing
      Train: Between Cities—Light Rail Transit—Streetcar (also known as Tram)—Rapid Rail Transit (also known as Metro or Subway
         On Platform
         Sitting
         Standing
   Airborne Vessel
      On Platform
      Sitting
      Standing
   Marine Vessel
      On Platform
      Sitting
      Standing

By the term “On Platform”, it is meant placing the portable device on a seat, table, or on dashboard or holder in case of car or bus.

FIG. 1 shows the motion modes and one possible set of categorizations in which the motion modes can be grouped or treated as a single motion mode. The problem of the determination of mode of motion or conveyance can: (i) tackle the lowest level of details directly, or (ii) can follow a divide and conquer scheme by tackling the highest level, then the middle level after one of the modes from highest level is determined, and finally the lowest level of details.

### The Process for Determining the Mode of Motion or Conveyance:

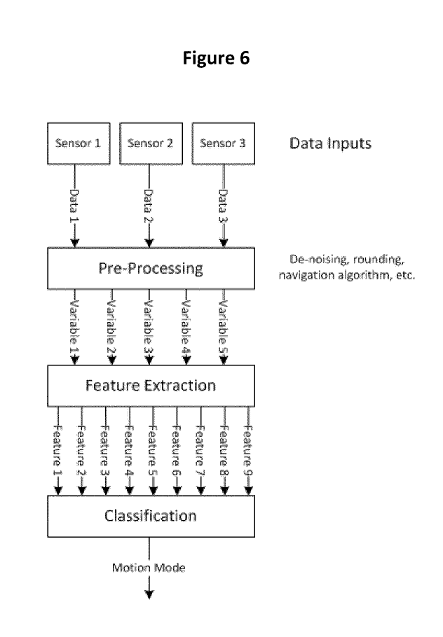

FIG. 6 explains the process to tackle the problem of motion mode recognition. An explanation for each step of the methodology shown is provided. The first step is obtaining some data inputs. The data inputs are obtained from the sensors from within the portable device. The data may be de-noised, rounded, or prepared it in a suitable condition for the successive steps.

The main two steps are feature extraction and classification. Feature extraction is the step needed to extract properties of the signal values which help discriminate different motion modes and it results in representing each sample or case by a feature vector: a group of features or values representing the sample or case. Feature selection and feature transformation can be used to help improve the feature vector. Classification is the process of determining the motion mode during a certain period given the feature values.

To build a classification model, as well as to build feature selection criteria and a feature transformation model, a training phase is needed where large amounts of training data need to be obtained. In the training phase, the model-building technique used can be any machine learning technique or any classification technique. Each model-building technique has its own methodology to generate a model which is supposed to obtain the best results for a given training data set. An evaluation phase follows the training phase, where evaluation data—data which have not been used in the training phase—are fed into the classification model and the output of the model, i.e., the predicted motion mode, is compared against the true motion mode to obtain an accuracy rate of the classification model.

### Data Inputs

The present method is used with a portable device which has the following sensors:
accelerometer triad:
   accelerometer in the x-axis, which measures specific force along the x-axis, fₓ,
   accelerometer in the y-axis, which measures specific force along the y-axis, f_{y},
   accelerometer in the z-axis, which measures specific force along the z-axis, f_{z},
gyroscope triad:
   gyroscope in the x-axis, which measures angular rotation rate along the x-axis, ωₓ,
   gyroscope in the y-axis, which measures angular rotation rate along the y-axis, ω_{y}, and
   gyroscope in the z-axis, which measures angular rotation rate along the z-axis, ω_{z}.

The device can also have the following optional sensors:
magnetometer triad:
   magnetometer in the x-axis, which measures magnetic field intensity along the x-axis,
   magnetometer in the y-axis, which measures magnetic field intensity along the y-axis,
   magnetometer in the z-axis, which measures magnetic field intensity along the z-axis, and
barometer, which measures barometric pressure and barometric height.

Using the readings from the sensors, and after applying any possible processing, fusing or de-noising, the following variables are calculated or estimated:
magnitude of leveled horizontal plane acceleration, aₕ: component of the acceleration of the device along the horizontal plane calculated in the local-level frame,
leveled vertical acceleration, aᵤₚ: vertical component of the acceleration of the device calculated in the local-level frame,
peaks detected on vertical leveled acceleration,
altitude or height, h: the height or altitude of the device measured above sea level or any pre-determined reference,
vertical velocity, vᵤₚ: the rate of change of height or altitude of the device with respect to time, and
norm of orthogonal rotation rates, |ω|: is the square root of the sum of squares of the rotation rates after subtracting their biases

|ω|=\sqrt{(ωₓ−b_{w}_{ₓ})²+(ω_{y}−b_{w}_{_{y}})²+(ω_{z}−b_{w}_{_{z}})²}

### Feature Extraction

In feature extraction, the above variables mentioned above for the last N, where N is any chosen positive integer, samples are obtained and a variety of features are extracted from them. The result of this operation is a feature vector: an array of values representing various features representing the window which includes the current sample and the previous N−1 samples.

Before extracting any of the features upon a variable, the variable may be rounded to a chosen precision, or the window of variables may be de-noised using a low pass filter or any de-noising methods.

Across each window of N samples, some or all of the following features may be extracted for each of the above mentioned variables, where u represents an array or vector of a variable with N elements:

### Statistical Features

### Mean of Values

Mean is a measure of the “middle” or “representative” value of a signal and is calculated by summing the values and dividing by the number of values:

$$mean ( u ) = \frac{1}{N} {\sum }_{n = 0}^{N - 1} u [ n ]$$

### Mean of Absolute of Values

The absolute of each value is taken first, i.e. any negative value is multiplied by −1, before taking the mean:

$$abs ( mean ( u ) ) = \overline{u [ n ]} = \frac{1}{N} {\sum }_{n = 0}^{N - 1} \vert u [ n ] \vert$$

### Median of Values

The median is the middle value of the signal values after ordering them in ascending order.

### Mode of Values

The mode is the most frequent value in the signal.

### 75^{th} Percentile of Values

Percentile is the value below which a certain percentage of the signal values fall. For example, the median is considered the 50% percentile. Therefore, 75 percentile is obtained by arranging the values in ascending order and choosing the [0.75N]^{th} value.

### Inter-Quartile Range of Values

Interquartile range is the difference between the 75 percentile and the 25 percentile.

### Variance of Values

Variance is an indicator of how much a signal is dispersed around its mean. It is equivalent to the mean of the squares of the differences between the signal values and their mean:

var(u)=σᵤ²= \overline{(u−\overline{u})²}

### Standard Deviation of Values

Standard deviation, σₓ, is the square root of the variance.

### Average Absolute Difference of Values

Average absolute difference is similar to variance. It is the average of the absolute values—rather than the squares—of the differences between the signal values and their mean:

AAD(u)= \overline{|u−\overline{u}|}

### Kurtosis of Values

Kurtosis is measure of the “peakedness” of the probability distribution of a signal, and is define by:

$$kurtosis ( u ) = \frac{\frac{1}{N} {\sum }_{n = 0}^{N - 1} {( u [ n ] - \overline{u} )}^{4}}{{( \frac{1}{N} {\sum }_{n = 0}^{N - 1} {( u [ n ] - \overline{u} )}^{2} )}^{2}}$$

### Skewness of Values

Skewness is measure of the asymmetry of the probability distribution of a signal, and is define by:

$$skewness ( u ) = \frac{\frac{1}{N} {\sum }_{n = 0}^{N - 1} {( u [ n ] - \overline{u} )}^{3}}{{( \sqrt{\frac{1}{N} {\sum }_{n = 0}^{N - 1} {( u [ n ] - \overline{u} )}^{2}} )}^{3}}$$

### Bin Distribution of Values

Binned distribution is obtained by dividing the possible values of a signal into different bins, each bin being a range between two values. The binned distribution is then a vector containing the number of values falling into the different bins.

### Time-Domain Features

The following features are concerned with the relation between the signal values and time.

### Zero-Crossing Rate of Values

Zero-crossing rate is the rate of sign change of the signal value, i.e. the rate of the signal value crossing the zero border. It may be mathematically expressed as:

$$zcr ( u ) = \frac{1}{N - 1} {\sum }_{n = 1}^{N - 1} I \{ u [ n ] u [ n - 1 ] < 0 \}$$

where I is the indicator function, which returns 1 if its argument is true and returns 0 if its argument is false.

### Number of Peaks of Values

Peaks may be obtained mathematically by looking for points at which the first derivative changes from a positive value to a negative value. To reduce the effect of noise, a threshold may be set on the value of the peak or on the derivate at the value of the peak. If there are no peaks meeting this threshold in a window, the threshold may be reduced until three peaks are found within the window.

### Energy, Magnitude, and Power Features

### Energy of Values

Signal energy refers to the square of the magnitude of the signal, and in our context, it refers to the sum of the squares of the signal magnitudes over the window.

$$energy ( u ) = {\sum }_{n = 0}^{N - 1} {( u [ n ] )}^{2}$$

### Sub-Band Energy of Values

Sub-band energy involves separating a signal into various sub-bands depending on its frequency components, for example by using band-pass filters, and then obtaining the energy of each band.

### Sub-Band Energy Ratio of Values

This is represented by the ratio of energies between each two sub-bands.

### Signal Magnitude Area of Values

Signal magnitude area (SMA) is the average of the absolute values of a signal:

$$SMA ( u ) = \frac{1}{N} {\sum }_{n = 0}^{N - 1} \vert u [ n ] \vert$$

### Frequency-Domain Features

Short-Time Fourier Transform (STFT), also known as Windowed Discrete Fourier Transform (WDFT), is simply a group of Fourier Transforms of a signal across windows of the signals:

$$STFT ( u ) [ k , m ] = {\sum }_{∀ n} u [ n ] w [ n - m ] {e}^{- j \frac{2 π kn}{N}}$$

$$where \text{:}$$

$$w [ n ] = \{ \begin{matrix}1 & if 0 \leq n \leq N - 1 \\ 0 & otherwise\end{matrix}$$

The result is a vector of complex values for each window representing the amplitudes of each frequency component of the values in the window. The length of the vector is equivalent to NFFT, the resolution of the Fourier transform operation, which can be any positive integer.

### Absolute of Short-Time Fourier Transform of Values

This is simply the absolute values of the output of short-time Fourier transform.

### Power of Short-Time Fourier Transform of Values

This is simply the square of the absolute values of the output of short-time Fourier transform.

### Power Spectral Centroid of Values

Power spectrum centroid is the centre point of the spectral density function of the signal of values, i.e., it is the point at which the area of the power spectral density plot is separated into 2 halves of equal area. It is expressed mathematically as:

$$SC ( u ) = \frac{{\sum }_{∀ f} fU ( f )}{{\sum }_{∀ f} U ( f )}$$

where U(f) is the Fourier transform of a signal u[n].

### Wavelet Transform of Values

Wavelet analysis is based on a windowing technique with variable-sized regions. Wavelet analysis allows the use of long time intervals where precise low frequency information is needed, and shorter intervals where high frequency information is considered. Either the continuous-time wavelet transform or discrete-time wavelet transform. For example, the continuous-time wavelet transform is expressed mathematically as:

$${CWT}_{ψ} ( u ) = \frac{1}{\sqrt{a}} {\int }_{- ∞}^{∞} u ( τ ) ψ ( \frac{t - τ}{a} ) d u$$

For each scale a and position τ, the time domain signal is multiplied by the wavelet function, ψ(t). The integration over time give the wavelet coefficient that corresponds to this scale a and this position τ.

The basis function, ψ(t), is not limited to exponential function. The only restriction on ψ(t) is that it must be short and oscillatory: it must have zero average and decay quickly at both ends.

After applying the wavelet transform and obtaining the output for each scale value, an operation may be obtained on each scale output, e.g., mean average, to obtain a vector representing the window.

### Spectral Fast Orthogonal Search Decomposition

Fast Orthogonal Search (FOS) with sinusoidal candidates can be used to obtain a more concise frequency analysis. Using this method, a system can be represented as:

$$u [ n ] = {\sum }_{i = 0}^{I} ( {b}_{i} cos {ω}_{i} n + {c}_{i} sin {ω}_{i} n ) + e [ n ]$$

where e[n] is the model error, and the frequencies ωᵢ need not be integer multiples of the fundamental frequency of the system, and therefore it is different to Fourier analysis. Fast orthogonal search may perform frequency analysis with higher resolution and less spectral leakage than Fast Fourier Transform (FFT) used over windowed data in STFT. Using this method, the most contributing M frequency components obtained from spectral FOS decomposition, and/or the amplitude of most contributing M frequency components obtained from spectral FOS decomposition, can be used as a feature vector, where M is an arbitrarily chosen positive integer.

### Frequency-Domain Entropy of Values

In information theory, the term entropy is a measure of the amount of information there is in a data set: the more diverse the values are within a data set, the more the entropy, and vice versa. The entropy of the frequency response of a signal is a measure of how much some frequency components are dominant. It is expressed mathematically as:

$${H}_{frequency \text{-} domain} = {\sum }_{i = 0}^{N} {P}_{i} log ( \frac{1}{{P}_{i}} )$$

where Pᵢ denotes the probability of each frequency component and is expressed as:

$${P}_{i} = \frac{\vert U ( {f}_{i} ) \vert}{{\sum }_{i = 0}^{N} \vert U ( {f}_{i} ) \vert}$$

where f is frequency and U(fᵢ) is the value of the signal x in the frequency domain, obtained by STFT, spectral FOS, or any other frequency analysis method.

### Other

### Cross-Correlation

Cross-correlation is a measure of the similarity between two signals as a function of the time lag between them. Cross-correlation between two signals may be expressed as a coefficient, which is a scalar, or as a sequence, which is a vector with length equal to the sum of the lengths of the two signals minus 1.

An example of cross-correlation coefficient is Pearson's cross-correlation coefficient, which is expressed as:

$${r}_{{u}_{1} {u}_{2}} = \frac{{\sum }_{n = 0}^{N - 1} ( {u}_{1} [ n ] - \overline{{u}_{1}} ) ( {u}_{2} [ n ] - \overline{{u}_{2}} )}{\sqrt{{\sum }_{n = 0}^{N - 1} {( {u}_{1} [ n ] - \overline{{u}_{1}} )}^{2} {\sum }_{n = 0}^{N - 1} {( {u}_{2} [ n ] - \overline{{u}_{2}} )}^{2}}}$$

where rᵤ_{₁}ᵤ_{₂} is Pearson's cross-correlation coefficient of signals u₁ and u₂.

The cross-correlation of values any two variables, e.g., leveled vertical acceleration versus leveled horizontal acceleration, can be a feature.

### Variable-to-Variable Ratio

Ratio of values of two variables, or two features, can be a feature in itself, e.g., average vertical velocity to number of peaks of leveled vertical acceleration in the window, or net change in altitude to number of peaks of leveled vertical acceleration in the window.

### Feature Selection and Transformation

After feature extraction, feature selection methods and feature transformation methods may be used to obtain a better feature vector for classification.

Feature selection aims to choose the most suitable subset of features. Feature selection methods can be multi-linear regression or non-linear analysis, which can be used to generate a model mapping feature extraction vector elements to motion mode output, and the most contributing elements in the model are selected. Non-linear or multi-linear regression methods may be fast orthogonal search (FOS) with polynomial candidates, or parallel cascade identification (PCI).

Feature transformation aims to obtain a mathematical transformation of the feature vector to create a new feature vector which is better and more representable of the motion mode. Feature transformation methods can be principal component analysis (PCI), factor analysis, and non-negative matrix factorization.

The feature selection criteria and feature transformation model are generated during the training phase.

### Classification

In classification, the feature vector is fed into a previously generated classification model whose output is one of the classes, where classes are the list of motion modes or categories of motion modes. The generation of the model may use any machine learning technique or any classification technique. The classification model detects the most likely motion mode which has been performed by the user of the device in the previous window. The classification model can also output the probability of each motion mode.

One or some or a combination of the following classification methods may be used:

### Threshold Analysis

This method simply compares a feature value with a threshold value: if it is larger or smaller than it then a certain motion mode is detected. A method named Receiver Operating Characteristic (ROC) can be used to obtain the best threshold value to discriminate two classes or motion modes from each other.

### Bayesian Classifiers

Bayesian classifiers employ Bayesian theorem, which relates the statistical and probability distribution of feature vector values to classes in order to obtain the probability of each class given a certain feature vector as input.

k-Nearest Neighbour

In this classification method, feature vectors are grouped into clusters, during the training phase, each corresponding to a class. Given an input feature vector, the cluster which is closest to this vector is considered to belong to that class.

### Decision Tree

A decision tree is a series of questions, with “yes” or “no” answers, which narrow down the possible classes until the most probable class is reached. It is represented graphically using a tree structure where each internal node is a test on one or more features, and the leaves refer to the decided classes.

In generating a decision tree, several options may be given to modify its performance, such as providing a cost matrix, which specifies the cost of misclassifying one class as another class, or providing a weight vector, which gives different weights to different training samples.

### Random Forest

Random forest is actually an ensemble or meta-level classifier, but it has proven to be one of the most accurate classification techniques. It consists of many decision trees, each decision tree classifying a subset of the data, and each node of each decision tree evaluates a randomly chosen subset of the features. In evaluating a new data sample, all the decision trees attempt to classify the new data sample and the chosen class is the class with highest votes amongst the results of each decision tree.

It is useful in handling data sets with large number of features, or unbalanced data sets, or data sets with missing data. It works better on categorical rather than continuous features. However, it may sometimes suffer from over-fitting if dealing with noisy data. Its resulting trees are difficult to interpret by humans, unlike decision trees. Random forests tend to bias towards categorical features with more levels over categorical features with fewer levels.

### Artificial Neural Networks

Artificial neural network (ANN) is a massively parallel distributed processor that allows pattern recognition and modeling of highly complex and non-linear problems with stochastic nature that cannot be solved using conventional algorithmic approaches.

### Fuzzy Inference System

Fuzzy inference system tries to define fuzzy membership functions to feature vector variables and classes and deduce fuzzy rules to relate feature vector inputs to classes. A neuro-fuzzy system attempts to use artificial neural networks to obtain fuzzy membership functions and fuzzy rules.

### Hidden Markov Model

A hidden Markov model aims to predict the class at an epoch by looking at both the feature vectors and at previously detected epochs by deducing conditional probabilities relating classes to feature vectors and transition probabilities relating a class at one epoch to a class at a previous epoch.

### Support Vector Machine

The idea of Support Vector Machine (SVM) is to find a “sphere” that contains most of the data corresponding to a class such that the sphere's radius can be minimized.

### Regression Analysis

Regression analysis refers to the set of many techniques to find the relationship between input and output. Logistic regression refers to regression analysis where output is categorical (i.e., can only take a set of values). Regression analysis can be, but not confined to, the following methods:
Linear Discriminant Analysis
Fast Orthogonal Search
Principal Component Analysis

### Post-Classification Methods

The results of classification may be further processed to enhance the probability of their correctness. This can be either done by smoothing the output—by averaging or using Hidden Markov Model—or by using meta-level classifiers.

### Output Averaging

Sudden and short transition from one class to another and back again to the same class, found in the classification output, may be reduced or removed by averaging, or choosing the mode of, the class output at each epoch with the class outputs of previous epochs.

### Hidden Markov Model

Hidden Markov Model can be used to smooth the output of a classifier. The observations of the HMM in this case are the outputs of the classifier rather than the feature inputs. The state-transition matrix is obtained from training data of a group of people over a whole week, while the emission matrix is set to be equal to the confusion matrix of the classifier.

### Meta-Level Classifiers

Meta-classifiers or ensemble classifiers are methods where several classifiers, of the same type or of different types, are trained over the same training data set, or trained on different subsets of the training data set, or trained using other classifier outputs treated as additional features, then evaluated, and their results combined in various ways. A combination of the output of more than one classifier can be done using the following meta-classifiers:
Voting: the result of each classifier is considered as a vote. The result with most votes wins. There are different modifications of voting meta-classifiers that can be used:
   Boosting: involves obtaining a weighted sum of the outputs of different classifiers to be the final output,
   Bagging (acronym for Bootstrap AGGregatING), the same classifier is trained over subsets of the original data, each subset is created as a random selection with replacement of the original data, and
   Plurality Voting: different classifiers are applied to the data and the output with highest vote is chosen.
Stacking: a learning technique is used to obtain the best way to combine the results of the different classifiers. Different methods that can be used are:
   stacking with ordinary-decision trees (ODTs): deduces a decision tree which decides the output class according to the outputs of the various classifiers,
   stacking with meta-decision trees (MDTs): deduces a decision tree which decides which classifier to be used according to the input,
   stacking using multi-response linear regression, and
Cascading: the output of a classifier is added as a feature to the feature set of another classifier.

### Example 2

### Building a Model for Determining the Mode of Motion or Conveyance

The following Example 2 provides a demonstrative example about how the classification model has been generated by collecting training data.

### Prototype

A low-cost prototype unit was used for collecting the sensors readings to build the model. Although the present method and system does not need all the sensors and systems in this prototype unit, they are mentioned in this example just to explain the prototype used. A low-cost prototype unit consisting of a six degrees of freedom inertial unit from Invensense (i.e. tri-axial gyroscopes and tri-axial accelerometer) (MPU-6050), tri-axial magnetometers from Honeywell (HMC5883L), barometer from Measurement Specialties (MS5803), and a GPS receiver from u-blox (LEA-5T) was used.

The axes frame of the example prototype is shown in Figure.

### Data Collection

A data collection phase was needed to collect training and evaluation data to generate the classification model. Using different instances of the prototype mentioned above with data logging software, many users, of various genders, ages, heights, weights, fitness levels, and motion styles, were asked to perform the motion modes mentioned in the previous example. Furthermore multiple different vessels with different features where used in those modes that involve such vessels. In order to generate robust classification models, users were asked to repeat each motion mode using different uses cases and different orientations. The uses cases covered in the tests were:
handheld (texting),
hand still by side of body,
dangling,
ear,
pocket,
belt,
chest,
arm,
leg,
wrist/watch,
on seat,
backpack,
purse,
glasses/head mount,
on seat, and
car holder.

### Processing

The variables mentioned above were obtained in one embodiment from a navigation solution within the portable device which fuses the readings from different sensors. At each epoch, the following features were then extracted from the windows of variables of length 64 samples:
mean of magnitude leveled horizontal plane acceleration,
mean of leveled vertical acceleration,
mean of norm of orthogonal rotation rates,
median of leveled horizontal plane acceleration,
median of leveled vertical acceleration,
median of norm of orthogonal rotation rates,
mode of magnitude leveled horizontal plane acceleration,
mode of leveled vertical acceleration,
mode of norm of orthogonal rotation rates,
75^{th} percentile of magnitude leveled horizontal plane acceleration,
75^{th} percentile of leveled vertical acceleration,
75^{th} percentile of norm of orthogonal rotation rates,
variance of magnitude leveled horizontal plane acceleration,
variance of leveled vertical acceleration,
variance of norm of orthogonal rotation rates,
variance of vertical velocity,
standard deviation of magnitude leveled horizontal plane acceleration,
standard deviation of leveled vertical acceleration,
standard deviation of norm of orthogonal rotation rates,
standard deviation of vertical velocity,
average absolute difference of magnitude leveled horizontal plane acceleration,
average absolute difference of leveled vertical acceleration,
average absolute difference of norm of orthogonal rotation rates,
inter-quartile range of magnitude leveled horizontal plane acceleration,
inter-quartile range of leveled vertical acceleration,
inter-quartile range of norm of orthogonal rotation rates,
skewness of magnitude leveled horizontal plane acceleration,
skewness of leveled vertical acceleration,
skewness of norm of orthogonal rotation rates,
kurtosis of magnitude leveled horizontal plane acceleration,
kurtosis of leveled vertical acceleration,
kurtosis of norm of orthogonal rotation rates,
binned distribution of magnitude leveled horizontal plane acceleration,
binned distribution of leveled vertical acceleration,
binned distribution of norm of orthogonal rotation rates,
energy of magnitude leveled horizontal plane acceleration,
energy of leveled vertical acceleration,
energy of norm of orthogonal rotation rates,
sub-band energy of magnitude leveled horizontal plane acceleration,
sub-band energy of leveled vertical acceleration,
sub-band energy of norm of orthogonal rotation rates,
sub-band energy of vertical velocity,
sub-band energy ratios of magnitude leveled horizontal plane acceleration,
sub-band energy ratios of leveled vertical acceleration,
sub-band energy ratios of norm of orthogonal rotation rates,
sub-band energy ratios of vertical velocity,
signal magnitude area of magnitude leveled horizontal plane acceleration,
signal magnitude area of leveled vertical acceleration,
signal magnitude area of norm of orthogonal rotation rates,
absolute value of short-time Fourier transform of magnitude leveled horizontal plane acceleration,
power of short-time Fourier transform of magnitude leveled horizontal plane acceleration,
absolute value of short-time Fourier transform of leveled vertical acceleration,
power of short-time Fourier transform of leveled vertical acceleration,
absolute value of short-time Fourier transform of norm of orthogonal rotation rates,
power of short-time Fourier transform of norm of orthogonal rotation rates,
absolute value of short-time Fourier transform of vertical velocity,
power of short-time Fourier transform of vertical velocity,
spectral power centroid of magnitude leveled horizontal plane acceleration,
spectral power centroid of leveled vertical acceleration,
spectral power centroid of norm of orthogonal rotation rates,
spectral power centroid of vertical velocity,
average of continuous wavelet transform of magnitude leveled horizontal plane acceleration,
average of continuous wavelet transform of leveled vertical acceleration,
average of continuous wavelet transform of norm of orthogonal rotation rates,
average of continuous wavelet transform of vertical velocity,
frequency entropy of magnitude leveled horizontal plane acceleration,
frequency entropy of leveled vertical acceleration,
frequency entropy of norm of orthogonal rotation rates,
frequency entropy of vertical velocity,
frequencies of the most contributing 4 frequency components of magnitude leveled horizontal plane acceleration,
amplitudes of the most contributing 4 frequency components of magnitude leveled horizontal plane acceleration,
frequencies of the most contributing 4 frequency components of leveled vertical acceleration,
amplitudes of the most contributing 4 frequency components of leveled vertical acceleration,
frequencies of the most contributing 4 frequency components of norm of orthogonal rotation rates,
amplitudes of the most contributing 4 frequency components of norm of orthogonal rotation rates,
average vertical velocity,
average of absolute of vertical velocity,
zero crossing rate of leveled vertical acceleration,
number of peaks of magnitude leveled horizontal plane acceleration,
number of peaks of leveled vertical acceleration,
number of peaks of leveled vertical acceleration,
cross-correlation of magnitude leveled horizontal plane acceleration versus leveled vertical acceleration,
ratio of vertical velocity to number of peaks of leveled vertical acceleration, and
ratio of net change of height to number of peaks of leveled vertical acceleration.

The classification method used was decision trees. A portion of the collected data was used to train the decision tree model, and the other portion was used to evaluate it.

In the coming examples, the present method and system are tested through a large number of trajectories from different modes of motion or conveyance including a large number of different use cases to demonstrate how the present method and system can handle different scenarios.

### Example 3

### Usage of the Classifier Model to Determine Height Changing Modes

### Example 3a

### Height Changing Motion Modes

This example illustrates a classification model to detect the following motion modes:
Stairs (categorizing Upstairs and Downstairs as one motion mode),
Elevator (categorizing Elevator Up and Elevator Down as one motion mode),
Escalator Standing (categorizing Escalator Up Standing and Escalator Down Standing), and
Escalator Walking (categorizing Escalator Up Walking and Escalator Down Walking).

A huge number of trajectories were collected by a lot of different people, using the prototypes in a large number of use cases and different orientations. About 700 trajectories were collected, with a total time for the height changing modes (stairs, elevator, escalator standing, escalator walking) of near 5 hours. Some of these datasets were used for model building and some for verification and evaluation.

All these trajectories contained different use cases and orientations including handheld (in any orientation or tilt), hand still by side of body, dangling, ear, pocket, belt, chest, arm, wrist, backpack, and purse.

Table 2 shows the confusion matrix of the evaluation results (using the evaluation data not included in the model building) of the decision tree model generated for this set of motion modes. The values in the table cells are percentage values. The average correction rate of the classifier was 89.54%. The results show that there was considerable misclassification between Escalator Moving and Stairs, which seems to be logical due to the resemblance between the 2 motion modes.

### Example 3b

### Height Changing Motion Modes Separated

Another approach is for the module to perform some logical checks to detect whether there are consecutive steps, and therefore decide whether to call one of two classification models. The same trajectories described above are used here, with all there uses cases as well.

The first classification model, whose confusion matrix (using the evaluation data not included in the model building) is shown in Table 3, discriminates height changing modes with steps, namely:
Stairs and
Escalator Walking

It has an average accuracy of 84.24% with higher misclassification for Escalator Walking.

The second classification model, whose confusion matrix (using the evaluation data not included in the model building) is shown in Table 4, discriminates height changing modes without steps, namely:
Elevator and
Escalator Standing

It has an average accuracy of 95.19%.

### Example 4

### Usage of the Classifier Model to Determine Walking, Running, Cycling and Land-Based Vessel Motion Modes

This example illustrates a classification model to detect the following motion modes:
Walking,
Running/Jogging,
Bicycle, and
Land-based Vessel: categorizing Car, Bus, and Train (different types of train, light and subways), as a single motion mode.

A huge number of trajectories were collected by a lot of different people/bicycles/vessels, using the prototypes in a large number of use cases and different orientations. About 1000 trajectories were collected, with a total time for the different modes of near 200 hours. Some of these datasets were used for model building and some for verification and evaluation.

The walking trajectories contained different use cases and orientations including handheld (in any orientation or tilt), hand still by side of body, dangling, ear, pocket, belt, chest, arm, wrist/watch, glasses/head mount, backpack, and purse. The walking trajectories also covered different speeds such as slow, normal, fast, and very fast, as well as different dynamics and gaits from different people with different characteristics such as height, weight, gender, and age. The running/jogging trajectories contained different use cases and orientations including chest, arm, wrist/watch, leg, pocket, belt, backpack, handheld (in any orientation or tilt), dangling, and ear. The running/jogging trajectories also covered different speeds such as very slow, slow, normal, fast, very fast, and extremely fast, as well as different dynamics and gaits from different people with different characteristics such as height, weight, gender, and age. The cycling trajectories contained different use cases and orientations including chest, arm, leg, pocket, belt, wrist/watch, backpack, mounted on thigh, attached to bicycle, and bicycle holder (in different locations on bicycle). The cycling trajectories also covered different people with different characteristics and different bicycles. The land-based vessel trajectories included car, bus, and train (different types of train, light rail, and subway), it also included sitting (in all vessel platforms), standing (in different types of trains and buses), and on platform (such as on seat in all vessel platforms, on car holder, on dashboard, in drawer, between seats); the uses cases in all the vessel platforms included pocket, belt, chest, ear, handheld, wrist/watch, glasses/head mounted, and backpack. The land-based vessels trajectories also covered for each type of vessel different instances with different characteristics and dynamics.

Table 5 shows the confusion matrix of the evaluation results (using the evaluation data not included in the model building) of the decision tree model generated for this set of motion modes. The values in the table cells are percentage values. The average correction rate of the classifier was 94.77%. Table 6 shows the confusion matrix of the evaluation results using the subset of evaluation data in GNSS-ignored trajectories (i.e. trajectories having GNSS ignored as if it is not available), to illustrate that the motion mode recognition module can work independently on GNSS availability, and the average correction rate was 93.825%.

### Example 5

### Usage of the Classifier Model to Determine Stationary or Non-Stationary Motion

This example illustrates a classification model o detect the following motion modes:
Stationary and
Non-Stationary.

A huge number of trajectories were collected by a lot of different people and vessels, using the prototypes in a large number of use cases and different orientations. More than 1400 trajectories were collected, with a total time of more than 240 hours. Some of these datasets were used for model building and some for verification and evaluation.

The non-stationary trajectories included all the previously mentioned trajectories of walking, running, cycling, land-based vessel, standing on a moving walkway, walking on a moving walkway, elevator, stairs, standing on an escalator, and walking on an escalator. As for the stationary mode, both ground stationary or in land-based vessel stationary were covered. By ground stationary, it is meant placing the device on a chair or a table, or on a person who is sitting or standing using handheld, hand still by side, pocket, ear, belt holder, arm band, chest, wrist, backpack, laptop bag, and head mount device usages. By land-based vessel stationary it is meant placing the device in a car, bus, or train, whose engines are turned on, with the device placed on the seat, dashboard, or cradle, or placed on the person who is either or sitting using the aforementioned device usages.

Table 7 shows the confusion matrix of the evaluation results (using the evaluation data not included in the model building) of the decision tree model generated for this set of motion modes. The values in the table cells are percentage values. The average correction rate of the classifier was 94.2%. Table 8 shows the confusion matrix of the evaluation results using the subset of evaluation data in GNSS-ignored trajectories (i.e. trajectories having GNSS ignored as if it is not available), to illustrate that the motion mode recognition module can work independently on GNSS availability and the average correction rate was 94.65%.

### Example 6

### Usage of the Classifier Model to Determine Standing or Walking on a Moving Walkway

This example illustrates classification models to detect the following motion modes:
Standing on Moving Walkway,
Stationary,
Walking on Moving Walkway, and
Walking (on ground).

A huge number of trajectories were collected by a lot of different people, using the prototypes in a large number of use cases and different orientations. More than 380 trajectories were collected for standing and walking on moving walkways, with a total time of near 10 hours. Some of these datasets were used for model building and some for verification and evaluation.

All these trajectories contained different use cases and orientations including handheld (in any orientation or tilt), hand still by side of body, dangling, ear, pocket, belt, chest, arm, wrist, backpack, and purse.

The first classification model, whose confusion matrix (using the evaluation data not included in the model building) is shown in Table 9, discriminates:
Stationary, and
Standing on Moving Walkway

It has an average accuracy of 84.2% with higher misclassification for Standing on Moving Walkway.

The second classification model, whose confusion matrix (using the evaluation data not included in the model building) is shown in Table 10, discriminates:
Walking and
Walking on a Moving Walkway
   It has an average accuracy of 73.1%.

### Example 7

### Usage of the Classifier Model to Determine Walking on Ground on or Walking within a Land-Based Vessel

This example illustrates classification models to detect the following motion modes:
Walking (on ground) and
Walking in Land-Based Vessel.

A huge number of trajectories were collected by a lot of different people, using the prototypes in a large number of use cases and different orientations. More than 1000 trajectories were collected for walking on ground and walking in land-based vessel, with a total time of near 20 hours. Some of these datasets were used for model building and some for verification and evaluation.

All these trajectories contained different use cases and orientations including handheld (in any orientation or tilt), hand still by side of body, dangling, ear, pocket, belt, chest, arm, wrist, backpack, and purse. The trajectories of walking in land-based vessel included walking in trains and walking in buses.

The classification model, whose confusion matrix (using the evaluation data not included in the model building) is shown in Table 11. It has an average accuracy of 82.5% with higher misclassification for Walking in Land-Based Vessel.

The embodiments and techniques described above may be implemented as a system or plurality of systems working in conjunction, or in software as various interconnected functional blocks or distinct software modules. This is not necessary, however, and there may be cases where these functional blocks or modules are equivalently aggregated into a single logic device, program or operation with unclear boundaries. In any event, the functional blocks and software modules implementing the embodiments described above, or features of the interface can be implemented by themselves, or in combination with other operations in either hardware or software, either within the device entirely, or in conjunction with the device and other processor enabled devices in communication with the device, such as a server.

Although a few embodiments have been shown and described, it will be appreciated by those skilled in the art that various changes and modifications can be made to these embodiments without changing or departing from their scope, intent or functionality. The terms and expressions used in the preceding specification have been used herein as terms of description and not of limitation, and there is no intention in the use of such terms and expressions of excluding equivalents of the features shown and described or portions thereof it being recognized that the invention is defined and limited only by the claims that follow.

### Appendix A

TABLE 1

| | | Sub-Motion |
|---|---|---|
| Category | Motion Mode | Mode |
| On Foot | Walking | |
| | Running/Jogging | |
| | Crawling | |
| | Fidgeting | |
| | Upstairs | |
| | Downstairs | |
| | Uphill | |
| | Downhill | |
| | Tilted Hill | |
| Cycling | | |
| Land-based | Car | On Platform |
| Vessel | | Sitting |
| | Bus: Within City-Between Cities | On Platform |
| | | Sitting |
| | | Standing |
| | | Walking |
| | Train: Between Cities-Light Rail | On Platform |
| | Transit-Streetcar-Rapid Rail Transit | Sitting |
| | | Standing |
| | | Walking |
| Airborne Vessel | | On Platform |
| | | Sitting |
| | | Standing |
| | | Walking |
| Marine Vessel | | On Platform |
| | | Sitting |
| | | Standing |
| | | Walking |
| On Foot within | Elevator Up | |
| Another | Elevator Down | |
| Platform | Escalator Up | Standing |
| | | Walking |
| | Escalator Down | Standing |
| | | Walking |
| | Conveyor Belt | Standing |
| | | Walking |
| Stationary | Ground | Sitting |
| | | Standing |
| | Bus: Within City-Between Cities | On Platform |
| | | Sitting |
| | | Standing |
| | Train: Between Cities-Light Rail | On Platform |
| | Transit-Streetcar-Rapid Rail Transit | Sitting |
| | | Standing |
| | AirborneVessel | On Platform |
| | | Sitting |
| | | Standing |
| | Marine Vessel | On Platform |
| | | Sitting |
| | | Standing |

### Appendix A

TABLE 2

| Actual | Determined Motion Mode | | | |
|---|---|---|---|---|
| Motion | | | Escalator | Escalator |
| Mode | Stairs | Elevator | Standing | Moving |
| Stairs | 89.3% | 0.3% | 0.1% | 10.3% |
| Elevator | 0.8% | 95.0% | 3.5% | 0.7% |
| Escalator | 0.6% | 5.5% | 93.6% | 0.3% |
| Standing | | | | |
| Escalator | 18.5% | 1.2% | 0.0% | 80.2% |
| Moving | | | | |

### Appendix A

TABLE 3

| | Determined Motion | |
|---|---|---|
| Actual | Mode | |
| Motion | | Escalator |
| Mode | Stairs | Moving |
| Stairs | 90.2% | 9.8% |
| Escalator | 21.7% | 78.3% |
| Moving | | |

### Appendix A

TABLE 4

| | Determined Motion | |
|---|---|---|
| Actual | Mode | |
| Motion | | Escalator |
| Mode | Elevator | Standing |
| Elevator | 96.2% | 3.8% |
| Escalator | 5.9% | 94.1% |
| Standing | | |

### Appendix A

TABLE 5

| Actual | Determined Motion Mode | | | |
|---|---|---|---|---|
| Motion | | | | Land-based |
| Mode | Walking | Running | Bicycle | Vessel |
| Walking | 96.5% | 2.2% | 1.1% | 0.3% |
| Running | 0.4% | 99.4% | 0.2% | 0.0% |
| Bicycle | 1.4% | 1.9% | 92.0% | 4.8% |
| Land-based | 0.3% | 0.0% | 8.4% | 91.2% |
| Vessel | | | | |

### Appendix A

TABLE 6

| Actual | Determined Motion Mode | | | |
|---|---|---|---|---|
| Motion | | | | Land-based |
| Mode | Walking | Running | Bicycle | Vessel |
| Walking | 95.2% | 0.7% | 4.0% | 0.2% |
| Running | 0.1% | 98.3% | 1.6% | 0.0% |
| Bicycle | 2.6% | 1.3% | 91.4% | 4.8% |
| Land-based | 1.7% | 0.1% | 7.8% | 90.4% |
| Vessel | | | | |

### Appendix A

TABLE 7

| Actual | | |
|---|---|---|
| Motion | Determined Motion Mode | |
| Mode | Stationary | Non-Stationary |
| Stationary | 90.5% | 9.5% |
| Non-Stationary | 2.1% | 97.9% |

### Appendix A

TABLE 8

| Actual | | |
|---|---|---|
| Motion | Determined Motion Mode | |
| Mode | Stationary | Non-Stationary |
| Stationary | 90.6% | 9.4% |
| Non-Stationary | 1.3% | 98.7% |

### Appendix A

TABLE 9

| Actual | | |
|---|---|---|
| Motion | Determined Motion Mode | |
| Mode | Stationary | Non-Stationary |
| Stationary | 97.2% | 2.8% |
| Standing on | 15.8% | 84.2% |
| Moving Walkway | | |

### Appendix A

TABLE 10

| Actual | | |
|---|---|---|
| Motion | Determined Motion Mode | |
| Mode | Stationary | Non-Stationary |
| Wlking | 90.2% | 9.8% |
| Walking on | 26.9% | 73.1% |
| Moving Walkway | | |

### Appendix A

TABLE 11

| | Determined Motion Mode | |
|---|---|---|
| Actual Motion | | Walking in |
| Mode | Walking | Land-Based Vessel |
| Walking | 91.4% | 8.6% |
| Walking in | 26.8% | 73.2% |
| Land-Based Vessel | | |

The embodiments in which an exclusive property or privilege is claimed are defined as follows:

1. A method for determining the mode of motion of a device, the device being within a platform and strapped or non-strapped to the platform, where non-strapped, mobility of the device may be constrained or unconstrained within the platform, the device having sensors capable of providing sensor readings, the method comprising the steps of:
a. obtaining features that represent motion dynamics or stationarity from the sensor readings; and
b. using the features to:
i. build a model capable of determining the mode of motion,
ii. utilize a model built to determine the mode of motion, or
iii. build a model capable of determining the mode of motion of the device, and utilizing said model built to determine the mode of motion.

2. A method for determining the mode of motion of a device, the device being within a platform and strapped or non-strapped to the platform, where non-strapped, mobility of the device may be constrained or unconstrained within the platform, the device having sensors capable of providing sensor readings, the method comprising the steps of:
a. obtaining the sensor readings for a plurality of modes of motion;
b. obtaining features that represent motion dynamics or stationarity from the sensor readings;
c. indicating reference modes of motion corresponding to the sensor readings and the features;
d. feeding the features and the reference modes of motion to a technique for building a model capable of determining the mode of motion; and
e. running the technique.

3. A method for determining the mode of motion of a device, the device being within a platform and strapped or non-strapped to the platform, where non-strapped, mobility of the device may be constrained or unconstrained within the platform, the device having sensors capable of providing sensor readings, the method comprising the steps of:
a. obtaining the sensor readings;
b. obtaining features that represent motion dynamics or stationarity from the sensor readings;
c. passing the features to a model capable of determining the mode of motion from the features; and
d. determining an output mode of motion from the model.

4. The method in any one of claims 1, 2, or 3, wherein the sensors comprise at least an accelerometer and at least a gyroscope.

5. The method in any one of claims 1, 2, or 3, wherein the sensors comprise at least a tri-axial accelerometer and at least a tri-axial gyroscope.

6. The method in claim 2, wherein the technique is a machine learning technique or a classification technique.

7. The method in claim 3, wherein the model is built using a machine learning technique.

8. The method in any one of claims 1, 2, or 3, wherein output of the model is a determination of the mode of motion.

9. The method in any one of claims 1, 2, or 3, wherein output of the model comprises determining the probability of each mode of motion.

10. The method in any one of claims 1, 2, or 3, wherein the method further comprises choosing a suitable subset of the features.

11. The method in any one of claims 1, 2, or 3, wherein the method further comprises a feature transformation step in order obtain the features better representing the mode of motion.

12. The method in any one of claims 1, 2, or 3, wherein the device further comprises a source of absolute navigational.

13. The method in any one of claims 1, 2, or 3, wherein a source of absolute navigational information is connected wirelessly or wired to the device.

14. The method in any one of claims 1 or 3, wherein the device further comprises a source of absolute navigational information, and wherein the method further comprises using absolute navigational information to further refine the determined mode of motion.

15. The method in any one of claims 1 or 3, wherein a source of absolute navigational information is connected wirelessly or wired to the device, and wherein the method further comprises using absolute navigational information to further refine the determined mode of motion.

16. The method in any one of claims 1 or 3, wherein the method further comprises refining the mode of motion based on a previous history of determined mode of motion.

17. The method of claim 16, wherein the refining is performed using filtering, averaging or smoothing.

18. The method of claim 16, wherein the refining is performed utilizing a majority of the previous history of determined mode of motion.

19. The method of claim 16, wherein the refining is performed utilizing hidden Markov Models.

20. The method in any one of claims 1, 2, or 3, wherein the method further comprises the use of meta-classification techniques, wherein a plurality of classifiers are trained and, when utilized, their results are combined to provide the determined mode of motion.

21. The method of claim 20, wherein the plurality of classifiers are trained on: (i) a same training data set, (ii) different subsets of the training data set, or (iii) using other classifier outputs as additional features.

22. A system for determining the mode of motion of a device, the device being within a platform, the system comprising:
a. the device strapped or non-strapped to the platform, where non-strapped, the mobility of the device may be constrained or unconstrained within the platform, the device comprising:
i. sensors capable of providing sensor readings; and
b. a processor programmed to receive the sensor readings, and operative to:
i. obtain features that represent motion dynamics or stationarity from the sensor readings; and
ii. use the features to: (A) build a model capable of determining the mode of motion, (B) utilize a model built to determine the mode of motion, or (C) build a model capable of determining the mode of motion and utilizing said model built to determine the mode of motion.

23. A system for determining the mode of motion of a device, the device being within a platform, the system comprising:
a. the device strapped or non-strapped to the platform, and where non-strapped, the mobility of the device may be constrained or unconstrained within the platform, the device comprising:
i. sensors capable of providing sensor readings; and
b. a processor operative to:
i. obtain the sensor readings for a plurality of modes of motion;
ii. obtain features that represent motion dynamics or stationarity from the sensor readings;
iii. indicate reference modes of motion corresponding to the sensor readings and the features;
iv. feed the features and the reference modes of motion to a technique for building a model capable of determining the mode of motion; and
v. run the technique.

24. A system for determining the mode of motion of a device, the device being within a platform, the system comprising:
a. the device strapped or non-strapped to the platform, where non-strapped, the mobility of the device may be constrained or unconstrained within the platform, the device comprising:
i. sensors capable of providing sensor readings; and
b. a processor operative to:
i. obtain the sensor readings;
ii. obtain features that represent motion dynamics or stationarity from the sensor readings;
iii. pass the features to a model capable of determining the mode of motion from the features; and
iv. determine an output mode of motion from the model.

25. The system in any one of claims 22, 23, or 24, wherein the sensors comprise at least an accelerometer and at least a gyroscope.

26. The system in any one of claims 22, 23, or 24, wherein the sensors comprise at least a tri-axial accelerometer and at least a tri-axial gyroscope.

27. The system in any one of claims 22, 22, or 24, wherein the device further comprises a source of absolute navigational information.

28. The system in any one of claims 22, 23, or 24, wherein a source of absolute navigational information is connected wirelessly or wired to the device.

29. The system of any one of claims 22, 23, or 24, wherein the processor is within the device.

30. The system of any one of claims 22, 23, or 24, wherein the processor is not within the device.
